SEQUENCE LISTING (32 sequences)

SEQ ID NO: 1 - DNA, 23 bp, Artificial Sequence
  EGFR Ex19 del C.I. 6210 forward primer
gtgagaaagttaaaattcccgtc

SEQ ID NO: 2 - DNA, 19 bp, Artificial Sequence
  EGFR Ex19 del C.I. 6210 reverse primer
cacacagcaaagcagaaac

SEQ ID NO: 3 - DNA, 23 bp, Artificial Sequence
  EGFR Ex19 del C.I. 6223 forward primer
gtgagaaagttaaaattcccgtc

SEQ ID NO: 4 - DNA, 19 bp, Artificial Sequence
  EGFR Ex19 del C.I. 6223 reverse primer
cacacagcaaagcagaaac

SEQ ID NO: 5 - DNA, 20 bp, Artificial Sequence
  KRAS G12A forward primer
gcctgctgaaaatgactgaa

SEQ ID NO: 6 - DNA, 20 bp, Artificial Sequence
  KRAS G12A reverse primer
gaatggtcctgcaccagtaa

SEQ ID NO: 7 - DNA, 20 bp, Artificial Sequence
  KRAS G12C forward primer
gcctgctgaaaatgactgaa

SEQ ID NO: 8 - DNA, 20 bp, Artificial Sequence
  KRAS G12C reverse primer
gaatggtcctgcaccagtaa

SEQ ID NO: 9 - DNA, 20 bp, Artificial Sequence
  KRAS G12D forward primer
gcctgctgaaaatgactgaa

SEQ ID NO: 10 - DNA, 20 bp, Artificial Sequence
  KRAS G12D reverse primer
gaatggtcctgcaccagtaa

SEQ ID NO: 11 - DNA, 20 bp, Artificial Sequence
  KRAS G12R forward primer
gcctgctgaaaatgactgaa

SEQ ID NO: 12 - DNA, 20 bp, Artificial Sequence
  KRAS G12R reverse primer
gaatggtcctgcaccagtaa

SEQ ID NO: 13 - DNA, 20 bp, Artificial Sequence
  KRAS G12S forward primer
gcctgctgaaaatgactgaa

SEQ ID NO: 14 - DNA, 20 bp, Artificial Sequence
  KRAS G12S reverse primer
gaatggtcctgcaccagtaa

SEQ ID NO: 15 - DNA, 20 bp, Artificial Sequence
  KRAS G12V forward primer
gcctgctgaaaatgactgaa

SEQ ID NO: 16 - DNA, 20 bp, Artificial Sequence
  KRAS G12V reverse primer
gaatggtcctgcaccagtaa

SEQ ID NO: 17 - DNA, 18 bp, Artificial Sequence
  EGFR Ex19 del C.I. 6210 PNA clamp N-->C
agatgttgcttctcttaa

SEQ ID NO: 18 - DNA, 18 bp, Artificial Sequence
  EGFR Ex19 del C.I. 6223 PNA clamp N-->C
agatgttgcttctcttaa

SEQ ID NO: 19 - DNA, 15 bp, Artificial Sequence
  KRAS G12A PNA clamp N-->C
tacgccaccagctcc

SEQ ID NO: 20 - DNA, 15 bp, Artificial Sequence
  KRAS G12C PNA clamp N-->C
tacgccaccagctcc

SEQ ID NO: 21 - DNA, 15 bp, Artificial Sequence
  KRAS G12D PNA clamp N-->C
tacgccaccagctcc

SEQ ID NO: 22 - DNA, 15 bp, Artificial Sequence
  KRAS G12R PNA clamp N-->C
tacgccaccagctcc

SEQ ID NO: 23 - DNA, 15 bp, Artificial Sequence
  KRAS G12S PNA clamp N-->C
tacgccaccagctcc

SEQ ID NO: 24 - DNA, 15 bp, Artificial Sequence
  KRAS G12V PNA sequence N-->C
tacgccaccagctcc

SEQ ID NO: 25 - DNA, 27 bp, Artificial Sequence
  EGFR Ex 19 del C.I. 6210 probe
ggctttcggagatgattccttgatagc

SEQ ID NO: 26 - DNA, 25 bp, Artificial Sequence
  EGFR Ex19 del C.I. 6223 probe
ctttcggagatgttttgatagcgac

SEQ ID NO: 27 - DNA, 21 bp, Artificial Sequence
  KRAS G12A probe
tgcctacgccagcagctccaa

SEQ ID NO: 28 - DNA, 21 bp, Artificial Sequence
  KRAS G12C probe
tgcctacgccacaagctccaa

SEQ ID NO: 29 - DNA, 21 bp, Artificial Sequence
  KRAS G12D probe
tgcctacgccatcagctccaa

SEQ ID NO: 30 - DNA, 21 bp, Artificial Sequence
  KRAS G12R probe
tgcctacgccacgagctccaa

SEQ ID NO: 31 - DNA, 21 bp, Artificial Sequence
  KRAS G12S probe
tgcctacgccactagctccaa

SEQ ID NO: 32 - DNA, 21 bp, Artificial Sequence
  KRAS G12V probe
tgcctacgccactagctccaa

(19) United States
(12) Patent Application Publication
(10) Pub. No.: US 2024/0392358 A1
(43) Pub. Date: Nov. 28, 2024

(54) Compositions and Methods for High Sensitivity Detection of Rare Mutations

(71) Applicant: CY Molecular Diagnostics, Inc., San Diego, CA (US)

(72) Inventors: William L. Hanna, San Diego, CA (US); Alexander Yum, San Diego, CA (US)

(21) Appl. No.: 18/035,850

(22) PCT Filed: Nov. 16, 2021
(86) PCT No.: PCT/US21/59537

§ 371 Date: May 8, 2023

Related U.S. Application Data
(60) Provisional application No. 63/114,957, filed on Nov. 17, 2020.

(51) Int. Cl. C12Q 1/6851; C12Q 1/6886

(52) U.S. Cl. CPC C12Q 1/6851; C12Q 1/6886; C12Q 2600/106; C12Q 2600/156

(57) ABSTRACT

Compositions and methods are described that provide a technique that reliably and robustly detects DNA mutations at present at concentrations as low as 0.001% relative to corresponding wild type DNA of the same DNA locus. Such compositions and methods are particularly suitable for clinical liquid biopsies, where cells containing diagnostically useful mutations are present in low numbers.

This application claims the benefit of U.S. Provisional Patent Application No. 63/114,957 filed on Nov. 17, 2020. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is detection of rare or low frequency mutations.

### BACKGROUND

The background description includes information that may be useful in understanding the present invention. It is not an admission that any of the information provided herein is prior art or relevant to the presently claimed invention, or that any publication specifically or implicitly referenced is prior art.

Methods for the detection of clinically relevant, small DNA mutations in the background of excess wild type DNA have formed the basis of modern companion diagnostics, for example companion diagnostics used to individualize treat methods in the administration of precision medicine oncology drugs. The presence or absence of specific DNA mutations in a patient's tumor are used to guide the administration of biologically targeted therapies that are selected on the basis of the specific genetic profile of the tumor. It is desirable to do so using a biopsy technique, in which a sample of tissue (e.g., tumor tissue, blood, etc.) is examined for the presence of tumor cells carrying specific mutations. Applications of traditional methods used to identify such mutations in such samples is, however, complicated by the presence of a large excess of wild type genetic material that have at least partial identity with the mutation.

Typical levels of DNA mutations as a percentage of the total DNA present in a biopsy obtained solid tumor biopsy sample can be high enough for conventional genotyping methods to reliably detect. For example, Sanger sequencing techniques can routinely detect DNA mutations as low as 15 % of the total DNA (i.e., 85 % wild type). Similar levels of sensitivity in detection of mutations are achieved by conventional PCR-based genotyping assays. However, with improved diagnostic techniques it has been shown that tissue biopsy tumor samples with clinically relevant DNA mutations below 15 % of the total DNA can also benefit from precision medicines. As a result of the need to identify lower percentages of DNA mutations in solid tumor biopsy samples, PCR genotyping and Sanger sequencing have been replaced by high sensitivity FDA-approved PCR-based and “next generation” sequencing-based techniques having mutation detection sensitivities ranging from 2 % to 8 %.

While the need for the detection of these low percentages challenge the assay developer, the mutation detection sensitivity needs of new liquid biopsy methods demand even higher sensitivity for mutation detection. Liquid biopsy samples are obtained from blood plasma, where materials originating from a patient's tumor are highly diluted and named circulating tumor DNA (ctDNA). In such samples DNA mutation levels can be in the range of 0.1 % or lower relative to wild type DNA, and often corresponding to fewer than 20 copies down to a single copy of mutant DNA per interrogated clinical sample. Examples of ultra-sensitive techniques used to date include digital PCR, new variations of next generation sequencing, and various forms of wild type suppression PCR. Each of these techniques, however, exhibits significant false positive and false negative events at the limits of their low-end range, especially when examining mutated tumor DNA below 20 copies per sample. While ultra-sensitive next generation sequencing (u-NGS) and Digital PCR (d-PCR) are each able to reproducibly identify mutated DNA down to 20 -25 and 10 -12 copies, respectively, each technique struggles as the mutated DNA copy levels fall below these levels, leading to uninterpretable or false negative results at these ultra-low mutant DNA copy levels.

The unmet need for even more sensitive mutant ctDNA detection methods has been prompted by three evolving categories of ctDNA cancer test, Minimal Residual Disease (MRD) detection, Resistance Monitoring (RM), and Early Cancer Detection (ECD). Since each of these clinical situations begins with zero or near-zero levels of mutated ctDNA and proceeds to shed more ctDNA into the plasma over time, each patient has a clinical window where mutant ctDNA copy levels are rising from zero to 10 or 20 copies per sample (generally correlating to 0.5 % mutant or less) where ctDNA mutants are unreliably detected by u-NGS and d-PCR. Therefore, there is a clinical need for technologies that robustly and reliably detect the earliest evidence of mutated ctDNA in the plasma (at levels between 1 and 10 copies per sample) to detect clinically addressable changes in existing tumors (i.e., MRD, RM) even earlier than existing assays. Perhaps even more importantly, such a technology could be employed to detect the lowest possible levels of mutant ctDNA in otherwise healthy patients for early cancer screening (or, ECD) where the Limit of Detection using u-NGS ctDNA mutant detection is typically greater than 30 copies per sample.

Thus, there is still a need for accurate and sensitive methods for identifying mutations occurring at very low frequencies, and particularly when mutant ctDNA levels fall between 1 and 10 copies per sample.

### SUMMARY OF THE INVENTION

The inventive subject matter provides apparatus, systems and methods using a combination of suppression of wild type sequence amplification and base-match sensitive luminescence, which provides a surprising synergistic effect that results in consistent and unambiguous (i.e., S/N ratio of 10 or more) detection of mutations present at low frequency (e.g., less than 0.1 %) relative to corresponding wild type genetic material present in the sample, and especially when the mutant DNA copy levels are below 10 per sample.

Embodiments of the inventive concept include a method of identifying a mutation by obtaining a sample (e.g., a sample that contains up to about 300 ng of human genomic DNA polynucleotide) that includes both a first polynucleotide that includes a wild type gene and a second polynucleotide that includes a mutation of the wide type gene (e.g., a deletion or a single nucleotide polymorphism, a transposition, a translocation, and/or an insertion), where the first polynucleotide is present in at least a 1,000 -fold excess over the second polynucleotide. An amplification reaction is then performed on the sample in which amplification of the first polynucleotide is at least partially suppressed (e.g., through the use of SNP discriminating clamps based on PNA, LNA, XNA, or other steric sequence-specific blockers like MGB clamps) and amplification of the second polynucleotide (containing the mutation) is not suppressed, using a primer pair complementary to both the first and second polynucleotides to generate an amplified sample that includes amplified first polynucleotide and an amplified second polynucleotide. This amplified sample is exposed to a probe sequence that includes a polynucleotide sequence complementary to at least a part of the amplified first polynucleotide and the amplified second polynucleotide, where the probe sequence includes an Click Chemistry modified acridinium-ester (i.e., SNP-Switch, Compound 25 from international patent application WO 2019/165469 A1 ) coupled to a linker that is positioned at or near the mutated site when the probe sequence is hybridized to the amplified second sequence. Light emission from the probe sequence is then measured, wherein the emission has a signal to noise ratio that is greater than 10 when at least one copy of the mutated sequence is present in the sample. In some embodiments the linker is coupled to a base of the probe sequence is complementary to the second polynucleotide, and includes a step of adding an oxidizing agent prior to measuring the emission. Suitable polynucleotides can include KRAS wild type or KRAS mutations (e.g., KRAS G12A, KRAS G12R, and KRAS G12V), and/or EGRF wild type and EGRF mutations (e.g., L858R, an Exon 19 deletion, a COSMIC 6223 mutation, and a COSMIC 6210 deletion). Suitable samples include those in which the first polynucleotide is present in at least a 10,000 fold excess and up to at least a 300,000 fold excess relative to the second polynucleotide. In some embodiments amplification is performed using a high fidelity polymerase.

Various objects, features, aspects and advantages of the inventive subject matter will become more apparent from the following detailed description of preferred embodiments, along with the accompanying drawing figures.

### BRIEF DESCRIPTION OF THE DRAWINGS

FIG. 1: FIG. 1 shows typical results from studies performed using samples containing 10 ng total DNA containing 0.1 % KRAS G12A mutation (mean of 3 copies per test well) relative to wild type DNA (99.9 %) using a method of the inventive concept.

FIG. 2: FIG. 2 shows typical results from studies of samples containing 10 ng total DNA containing the KRAS G12R mutation at 0.01 % (mean of 0.3 copies per test well) relative to corresponding wild type DNA in the sample (99.99 %) using a method of the inventive concept.

FIG. 3: FIG. 3 shows typical results of studies similar to those shown in FIG. 2, but performed using samples containing 10 ng total DNA containing 0.01 % KRAS G12A mutation (mean of 0.3 copies per test well) relative to corresponding wild type DNA content (99.99 %) using a method of the inventive concept.

FIG. 4: FIG. 4 shows typical results from studies similar to those shown in FIGS. 2 and 3, but were performed with samples containing 10 ng total DNA containing 0.01 % KRAS G12V mutation (mean of 0.3 copies per test well) relative to corresponding wild type DNA (99.99 %) using a method of the inventive concept.

FIG. 5: FIG. 5 shows typical results of studies performed using samples containing 10 ng total DNA containing the EGFR exon 19 deletion at 0.1 % (mean of 3 copies per test well) relative to corresponding wild type DNA (99.9 %) using a method of the inventive concept.

FIG. 6: FIG. 6 shows typical results from studies of samples containing 10 ng total DNA containing the EGFR Exon 19 Del C#6210 mutation at 0.01 % (mean of 0.3 copies per test well) relative to corresponding wild type DNA in the sample using a method of the inventive concept.

FIG. 7: FIG. 7 shows typical results of studies performed using samples containing 3 ng of DNA containing 0.1 % EGFR C6223 mutation (mean of 1 copy per well) relative to corresponding wild type DNA (99.9 %) using a method of the inventive concept.

FIG. 8: FIG. 8 shows typical results of tests performed using samples containing 3 ng of DNA containing 0.1 % KRAS G12A mutation (mean of 1 copy per well)) relative to corresponding wild type DNA (99.9 %) using a method of the inventive concept.

FIG. 9: FIG. 9 shows typical results of tests performed using samples containing 100 ng per test sample, where the samples contain 0.01 % KRAS G12A mutation (mean of 3 copies per well) relative to corresponding wild type DNA (99.99 %).

FIG. 10: FIG. 10 shows typical results of tests performed using samples containing 100 ng per test sample, where the samples contain KRAS G12A mutation at 0.001 % (mean of 0.3 copies per well) relative to corresponding wild type DNA (99.999 %) using a method of the inventive concept.

FIG. 11: FIG. 11 shows typical results of studies performed on samples containing 333 ng of DNA, which included 0.0003 % KRAS G12A mutation (mean of 0.3 copies per well) relative to corresponding wild type DNA (99.9997 %) using a method of the inventive concept.

FIG. 12: FIG. 12 shows results of methods of the inventive concept applied to different KRAS mutations. Panel A shows results obtained with a 3 ng sample containing KRAS G12C at 10, 1, and 0 copies, the remainder being wild type DNA. Panel B shows results obtained with a 10 ng sample containing KRAS G12D at 30, 3, and 0 copies, the remainder being wild type DNA. Panel C shows results with a 3 ng samples containing KRAS G12 S at 10, 1, and 0 copies, with the remainder being wild type DNA.

FIG. 13: FIG. 13 shows typical results for a method of the inventive concept directed towards detection of the PCR EGFR Ex19del mutation present at low copy numbers in unfragmented genomic DNA (gDNA) and fragmented DNA (cfDNA).

FIG. 14: FIG. 14 shows typical results for methods of the inventive concept applied to cell-free DNA samples obtained from blood. Panel A shows demographic data for 114 random blood bank donors used in the study. ‘Panel B shows typical results obtained from 10 ng samples DNA samples obtained from these donors as well as such samples to which 10 copies of EGRF Ex19 DelC6223 mutation had been added. No false positive or false negatives are noted. Panel C shows typical results of studies similar to those shown in Panel B, but utilizing the KRAS G12A mutation.

FIG. 15: FIG. 15 shows typical results of parallel studies of detection limit for a commercial KRAS mutation digital droplet PCR (ddPCR) kit and a method of the inventive concept directed to the KRAS mutation. Panel A of FIG. 15 shows results from the KRAS mutation channel of the ddPCR kit for 10 ng samples from a patient with the KRAS mutation that have been diluted with wild type DNA to 10 copies of 5 copies per sample. Panel B shows results from the KRAS wild type channel of the ddPCR kit. Panel C shows results from a KRAS mutation assay of the inventive concept for the samples shown in Panel A and also samples diluted to provide 1 copy of the KRAS mutation per sample.

FIG. 16: FIG. 16 provides a graphical summary of the data shown in FIG. 15.

### DETAILED DESCRIPTION

The inventive subject matter provides compositions and methods that provide a PCR-based technique that reliably and robustly (i.e., S/N ratio of 3.5 or more) detects rare DNA mutations (less than 0.1 % relative to corresponding wild type sequence in the sample) present at concentrations down to 0.01 % and 0.001 % relative to corresponding wild type DNA (i.e. corresponding to the same DNA locus but having the wild type genotype). Such compositions and methods are highly suitable for clinical liquid biopsies as they permit reproducible and accurate detection of samples containing as little as 1 to 20 copies of mutant DNA in the presence of a large excess (99.9 %, 99.99 %. 99.999 % or greater) of wild type DNA.

Various objects, features, aspects and advantages of the inventive subject matter will become more apparent from the following detailed description of preferred embodiments, along with the accompanying drawing figures in which like numerals represent like components.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

One should appreciate that the disclosed techniques provide many advantageous technical effects including improving the accuracy and sensitivity of relatively non-invasive liquid biopsies.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

One conventional method of improving detection of DNA containing mutations against a background of wild type DNA is to selectively suppress PCR amplification of the wild type sequence. Suppression of wild type DNA amplification can be accomplished through the use of wild type specific ‘clamps’ that interfere with replication of the wild type sequence. Such clamps can include non-naturally occurring nucleotides/nucleotide analogs and/or non-naturally occurring backbone structures. Examples of these include (but are not limited to) PNAs, LNAs, and/or XNA (Diacarta). In some embodiments suppression of wild type DNA can be accomplished by using a primer pair that includes at least one single base pair mismatch.

For example, a PNA that is complementary to the wild type sequence but does not act as a primer for the DNA polymerase can be used to suppress amplification of a wild type sequence. Such an approach can suppress replication in a limited fashion. Given a sufficient number of replication cycles nonspecific ‘background’ amplification renders amplification of mutated sequences undetectable. Inventors have found that the use of PNAs for suppression of wild type amplification can provide detectable amplification of mutated DNA present at as low as about 1 % relative to the corresponding wild type sequence, generally showing robust amplification after about 25 to 30 thermal cycles when using conventional methods visualized using SYBR green. This represents an improvement of approximately 10 -15 fold over conventional PCR-based methods.

Another conventional approach to improving detection of DNA containing mutations against a background of corresponding wild type DNA is to visualize amplification products using probes that are sensitive to base-pair mismatches, such as luminescence based probes. Results from these can be visualized as the signal: noise (S/N) ratio observed over background luminescence. Observation of experiments performed using only wild type DNA and mutant-specific chemiluminescent probes show frequent non-specific signals with a S/N ratio of about 2 to 2.5 -fold over background luminescence. Accordingly, a minimum S/N ratio of 3 to 3.5 is necessary to have assurance that the observed signal is due to the presence of mutant DNA. Inventors have found that luminescent mutant-specific probes typically provide a S/N ratio of from about 3 to 4 when mutant DNA is present at 3 % to 5 % relative to corresponding wild type DNA. However, this decreases rapidly as the percentage of mutant DNA decreases. Inventors have observed average S/N ratios ranging from about 1 to 2 when a large number of replicates at 1 % mutant DNA relative to corresponding wild type DNA are characterized using chemiluminescent probes, however this is too low for consistent and practical detection of the presence of a mutation. Overall, the use of chemiluminescent probes that are sensitive to base pair mismatches provides an improvement of approximately 3 -5 fold over conventional PCR-based methods.

Accordingly, combination of suppression of wild type DNA amplification and the use of chemiluminescent probes sensitive to base pair mismatches would be expected, in the absence of an unexpected synergistic effect, to provide an overall improvement in detections sensitivity for mutations of about 6 -15 fold, or to provide reliable detection of mutations at down to about 0.3 % mutant DNA content relative to corresponding wild type DNA present in the sample. Surprisingly, Inventors have found that such a combination (which within the context of this application is termed “ssPCR”) provides reliable (i.e. S/N>3.5 ) detection of mutant DNA at substantially less than 0.1 % relative to corresponding wild type DNA present in the sample when luminescence-based probes that are selective based on single base pair mismatches are used in concert with a PNA that selectively hybridizes to the corresponding wild type DNA sequence, indicating a significant and unexpected synergy between these methods. The effect is particularly marked at low copy numbers.

Accordingly, methods of the inventive concept utilize an unexpected synergistic effect of the combination of suppression of wild type sequence amplification (e.g., through the use of a clamping technique utilizing a PNA. LNA, and/or XNA primer complementary to wild type sequence) with base-pair mismatch specific labeling or detection to achieve simple and reliable detection of rare mutations at unexpectedly low frequencies relative to corresponding wild type DNA present in the sample. This greatly facilitates the sensitivity and/or reliability of relatively non-invasive liquid biopsies, which typically utilize a blood sample in which cancer cells containing mutations to be detected are present in a milieu of cells having normal genotype.

Suppression of the replication of wild type sequences that correspond to mutations of interest can be performed by any suitable means. These include, but are not limited to, the use of wild type specific primers that interfere with replication of the wild type sequence. Such primers can include non-naturally occurring nucleotides/nucleotide analogs and/or non-naturally occurring backbone structures. Examples of these include (but are not limited to) PNAs, LNAs, and/or XNA (Diacarta). For example, a PNA that is complementary to the wild type sequence but does not act as a primer for the DNA polymerase can be used to suppress amplification of a wild type sequence.

Ideally, methods used to suppress wild-type amplification should result in no discernible amplification within the amplification cycles utilized in the assay (e.g., 5, 10, 15, 20, 25, 30, 35, 40, 45, 50, 60, 70, 80, 90, 100, or more than 100 amplification cycles). Surprisingly, Inventors have found that ssPCR can provide high sensitivity results for detection of rare mutations over a background of a large (e.g., greater than 100 -fold, 1,00 -fold, or 10,000 -fold) excess of wild type DNA when suppression of wild-type amplification is incomplete (i.e., resulting in discernible amplification of wild-type sequence during the course of the assay).

Detection of base pair mismatches in amplified sequences can be performed by any suitable means in methods of the inventive concept. In some embodiments detection can be performed using a probe that incorporates one or more lanthanide-based luminescent compounds that exhibit mutation-specific luminescence. Although luminescent probe sequences are utilized in the examples provided below, it should be recognized that other detection methodologies where the reporting portion or method is responsive to the site of the mutation (i.e., provides a detectable response or signal, or change in response or signal, that is dependent on the presence of the specific mutation) can be used. Suitable methodologies include, but are not limited to, fluorescence, fluorescence polarization, Foerster Resonance Energy Transfer, and mass spectrometry (e.g., following digestion of amplification products with a suitable nuclease).

It should be appreciated that methods of the inventive concept can be used in the detection of a variety or rare mutations, including deletions and/or single nucleotide polymorphisms (SNPs). Such methods are referred to within the context of this application as SNP-switch PCR or ssPCR.

### Methods

DNA Samples: Human wild type genomic DNA pooled from multiple donors was obtained from Promega (p/n G3041 ), and was used in combination with human cell line genomic DNA containing a single and specific engineered DNA mutation in an otherwise wild type locus. Each of the engineered DNA samples arrived with a specific mutant frequency determined by each manufacturer using digital PCR. Engineered genomic DNA samples were 50 % mutant, and were serially diluted into pooled wild type human genomic DNA samples using a standardized process. Briefly, using the example of 10 ng DNA samples in 20 μl PCR reactions, low mutant percentage samples were generated by diluting concentrated wild type and mutated DNA stocks to 1 ng/μl and serially diluting engineered mutated DNA into wild type DNA in a series of 1:2 to 1:10 dilutions in conveniently measured volumes exceeding 3 μl per serial transfer. For example, to generate a 10 ng PCR sample, mutant DNA panels were serially diluted down to 1 % (30 mutant copies/10 μl), 0.1 % (3 copies/10 μl), and 0.01 % (0.3 copies/10 μl). In some experiments the PCR samples contained a total of 1 ng to 100 ng DNA, and mutant DNA percentage panels were generated in a similar fashion. Engineered mutant DNA samples are listed in the Table 1.

TABLE 1
ENGINEERED HUMAN CELL LINE GENOMIC DNA

| Gene | Mutant | COSMIC ID | Seracare Onco-Ref | Horizon p/n ¹ |
|---|---|---|---|---|
| EGFR | Ex19 del | 6210 | Aso-6019-1 | |
| EGFR | Ex19 del | 6223 | | HD251 |
| KRAS | G12A | 522 | | HD265 |
| KRAS | G12C | 516 | | HD269 |
| KRAS | G12D | 521 | | HD272 |
| KRAS | G12R | 518 | | HD287 |
| KRAS | G12S | 517 | | HD288 |
| KRAS | G12V | 520 | | HD289 |

¹ Horizon Discovery, UK

Standard PCR Amplification: Standard PCR amplification was performed in 96 well plates, on a ThermoFisher QuantStudio 5 thermal cycler. For each 20 μl PCR reaction, 1 -100 ng of human genomic DNA sample in 10 μl TE buffer (pH 8.0 ) primers were prepared using 9.5 μl of 2× PowerSYBR PCR Master Mix (ThermoFisher, p/n 4368577 ) with 0.5 μl of primers in TE buffer for a final primer concentration of 200 nM in a 20 μl PCR reaction. All PCRs followed the same program: initial DNA denaturation and polymerase activation at 95° C. for 10 minutes, followed by 40 cycles of 95° C. for 3 seconds, annealing at 57° C. for 30 seconds, extension at 72° C. for 15 seconds, and after cycling a final extension at 72° C. for 2 minutes. PCR primers are described in Table 2.

TABLE 2
ssPCR AMPLIFICATION PRIMERS

| | | | | SEQ | |
|---|---|---|---|---|---|
| | | COSMIC | | ID | |
| Gene | Mutant | ID | Primer | NO. | Sequence |
| EGFR | Ex19 | 6210 | Fwd | 1 | 5′GTGAGAAAGTTAAAATTCCCGTC3′ |
| | del | | Rev | 2 | 5′CACACAGCAAAGCAGAAAC3′ |
| EGFR | Ex19 | 6223 | Fwd | 3 | 5′GTGAGAAAGTTAAAATTCCCGTC3′ |
| | del | | Rev | 4 | 5′CACACAGCAAAGCAGAAAC3′ |
| KRAS | G12A | 522 | Fwd | 5 | 5′GCCTGCTGAAAATGACTGAA3′ |
| | | | Rev | 6 | 5′GAATGGTCCTGCACCAGTAA3′ |
| KRAS | G12C | 516 | Fwd | 7 | 5′GCCTGCTGAAAATGACTGAA3′ |
| | | | Rev | 8 | 5′GAATGGTCCTGCACCAGTAA3′ |
| KRAS | G12D | 521 | Fwd | 9 | 5′GCCTGCTGAAAATGACTGAA3′ |
| | | | Rev | 10 | 5′GAATGGTCCTGCACCAGTAA3′ |
| KRAS | G12R | 518 | Fwd | 11 | 5′GCCTGCTGAAAATGACTGAA3′ |
| | | | Rev | 12 | 5′GAATGGTCCTGCACCAGTAA3′ |
| KRAS | G12S | 517 | Fwd | 13 | 5′GCCTGCTGAAAATGACTGAA3′ |
| | | | Rev | 14 | 5′GAATGGTCCTGCACCAGTAA3′ |
| KRAS | G12V | 520 | Fwd | 15 | 5′GCCTGCTGAAAATGACTGAA3′ |
| | | | Rev | 16 | 5′GAATGGTCCTGCACCAGTAA3′ |

Wild type-Suppression PCR Amplification: For wild type-suppression PCR, the standard PCR format was followed, except for the addition of peptide nucleic acid (PNA) clamps (listed in Table 3 ) at a range of 1-100 pmole per 20 μl PCR reaction.

TABLE 3
ssPCR PEPTIDE NUCLEIC ACID CLAMPS

| | | COSMIC | SEQ | PNA Clamp |
|---|---|---|---|---|
| Gene | Mutant | ID | ID NO. | Nucleotide Sequence |
| EGFR | Ex19 | 6210 | 17 | N-AGATGTTGCTTCTCTTA |
| | del | | | A-C |
| EGFR | Ex19 | 6223 | 18 | N-AGATGTTGCTTCTCTTA |
| | del | | | A-C |
| KRAS | G12A | 522 | 19 | N-TACGCCACCAGCTCC-C |
| KRAS | G12C | 516 | 20 | N-TACGCCACCAGCTCC-C |
| KRAS | G12D | 521 | 21 | N-TACGCCACCAGCTCC-C |
| KRAS | G12R | 518 | 22 | N-TACGCCACCAGCTCC-C |
| KRAS | G12S | 517 | 23 | N-TACGCCACCAGCTCC-C |
| KRAS | G12V | 520 | 24 | N-TACGCCACCAGCTCC-C |

DNA Probes: DNA oligonucleotide probes were synthesized using phosphoramidite chemistry by Eton Biosciences, and was incorporated with an internal amine linker synthesized by Tenova Pharmaceuticals. Oligonucleotides were post-synthetically labeled by the addition of Click Chemistry-enhanced acridinium ester (SNP-Switch, Compound 25 from international patent application WO 2019/165469 A1 ), or in some cases 9 [[4-[3-[(2,5-dioxo-1-pyrrolidinyl)oxy]-3-oxopropyl]phenoxy]carbonyl]-10-methyl-acridinium, 1,1,1- trifluoromethanesulfonate. Each compound was synthesized by Teknova Pharmaceuticals. The SNP-Switch (Compound 25 ) was chemically attached to the oligonucleotide's free amine group. Labeling reactions consisted of 1 nmole of oligonucleotide in 3 μl H₂O, 4 μl DMSO, 1 μl of 1 M HEPES pH 8.0, and 2 μl of 25 mM or SNP-Switch (Compound 25 ) or in some cases 9 [[4-[3-[(2,5-dioxo-1-pyrrolidinyl) oxy]-3-oxopropyl] phenoxy] carbonyl]-10-methyl-acridinium, 1,1,1-trifluoromethanesulfonate in DMSO. The reaction mixture was incubated at 37° C. for 20 minutes, then mixed with 5 μl of 0.125 M L-Lysine in 0.1 M HEPES, pH 8.0, 50 % DMSO, and incubated at room temperature for 5 minutes. Following this 5 minute incubation, 30 μl of 3 M NaOAc, pH 5.0, 245 μl of DNase-free water, and 5 μl of molecular grade glycogen were first added, followed by an additional 640 μl of 100 % ethanol. This final reaction mixture was vortexed, then chilled at-20° C. for 10 minutes then centrifuged at 17,000× g for 5 minutes. Following centrifugation, the supernatant was removed, and the pellet was air-dried for 15 minutes. The pellet containing the labelled probe was solubilized in 1 ml of acidic buffer (10 mM succinic acid, pH 5.0, with 0.1 % lithium lauryl sulfate), and stored at −20° C. between uses. DNA probe solutions used in post-PCR endpoint assays were adjusted to between 0.05 pmole and 1 pmole per 100 μl of a 1:1 mixture of acidic annealing buffer (200 mM succinic acid, 10 % lithium lauryl sulfate, 0.8 M lithium chloride, 2 mM EDTA, pH 5.0 ) and acidic buffer. Probe sequences are listed in Table 4.

TABLE 4
ssPCR LUMINESCENT DNA PROBES

| | | COSMIC | SEQ | |
|---|---|---|---|---|
| Gene | Mutant | ID | ID NO. | DNA Probe Sequence |
| EGFR | Ex19 | 6210 | 25 | 5′GGCTTTCGGAGATG// |
| | del | | | ATTCCTTGATAGC3′ |
| EGFR | Ex19 | 6223 | 26 | 5′CTTTCGGAGATGTT// |
| | del | | | TTGATAGCGAC3′ |
| KRAS | G12A | 522 | 27 | 5′TGCCTACGCCA// |
| | | | | GCAGCTCCAA3′ |
| KRAS | G12C | 516 | 28 | 5′TGCCTACGCCACA// |
| | | | | AGCTCCAA3′ |
| KRAS | G12D | 521 | 29 | 5′TGCCTACGCCA// |
| | | | | TCAGCTCCAA3′ |
| KRAS | G12R | 518 | 30 | 5′TGCCTACGCCACG// |
| | | | | AGCTCCAA3′ |
| KRAS | G12S | 517 | 31 | 5′TGCCTACGCCAC// |
| | | | | TAGCTCCAA3′ |
| KRAS | G12V | 520 | 32 | 5′TGCCTACGCCAC// |
| | | | | TAGCTCCAA3′ |

Endpoint ssPCR Mutation Detection: Immediately following PCR, the plate was unsealed and 200 μl of DNA probe solution was added to the 96 well plate containing 20 μl PCR reaction volumes. The contents of each PCR well were transferred to a 5 ml polypropylene tube (12×75 mm), placed in a 5 ml tube-compatible heated vortexer pre-warmed to 95° C., briefly vortexed, and incubated for 1 minute. The heater was then adjusted to 60° C., and the tubes were incubated for another 10 minutes. Following this, 300 μl of alkaline shock buffer (150 mM sodium tetraborate, 0.5 % Triton X-100, pH 8.5 ) was added to each tube, vortexed, and incubated for another 20 to 60 minutes at 60° C.

Following the last incubation, the tubes are removed to room temperature for 3 minutes before being analyzed for residual luminescence in a dual injection tube luminometer, first injecting 300 μl of light solution 1 (1 mM nitric acid, 0.1 % H₂O₂ ), with a 1 second pause before the injection of 300 μl of light solution 2 (1.6 M sodium hydroxide) and then read for 2 seconds.

Using materials and methods as described above, a series of studies were performed for detection of a range of mutations provided at low frequency relative to corresponding wild type DNA in the sample. Samples containing a range of total DNA content were characterized using the PNA clamping technique described above to suppress wild type DNA replication and utilizing mutation-specific luminescence-based probes.

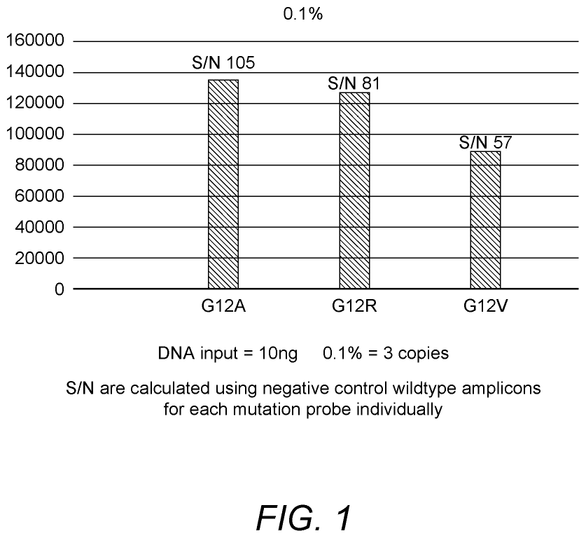

Results from ssPCR studies performed using samples containing 10 ng total DNA with 0.1 % KRAS G12A mutation relative to wild type DNA are shown in FIG. 1. It should be appreciated that with the volumes utilized this represents results from an average of 3 copies of the KRAS G12A, G12V, and G12R mutations in the samples. Results shown are the average of 3 test wells. Surprisingly, this implementation of the inventive concept provided S/N ratios >50 for all three mutations at a frequency of only 0.1 %.

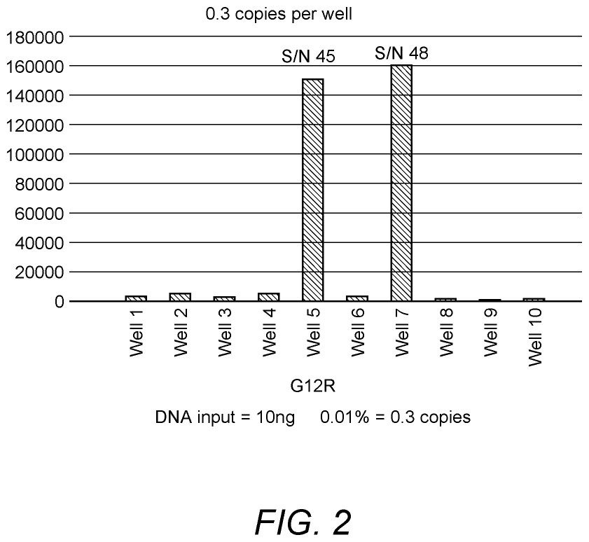

FIG. 2. shows results from ssPCR studies of samples containing the KRAS G12R mutation at 0.01 % relative to corresponding wild type DNA in the sample. Each sample containing 10 ng of DNA, providing a mutant copy number of 0.3 per well. Accordingly, it is to be expected that among the 10 wells tested many will not include any mutant DNA. As expected, under these conditions 8 of 10 test wells show only background luminescence, indicating that no mutant DNA is present. Surprisingly, two test wells show a clearly distinctive and detectable S/N ratio of nearly 50 from samples containing only a single copy of the mutation in the presence of a large excess (99.99 % of total DNA content) of wild type DNA.

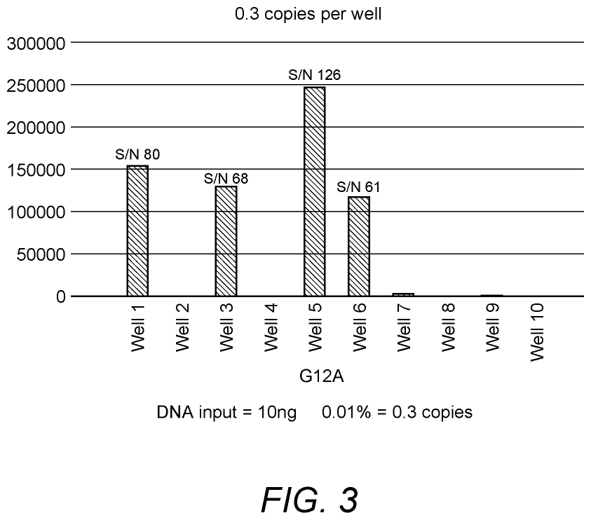

FIG. 3 shows the results of ssPCR studies similar to those shown in FIG. 2 (i.e., a mean of 0.3 copies of mutant DNA per test well), but performed using samples containing 0.01 % KRAS G12A mutation relative to corresponding wild type DNA content. Under these conditions 5 of 10 test wells show only background luminescence, indicating that no mutant DNA is present. Three test wells show a clearly distinctive and detectable S/N ratio of greater than 60 from samples containing only a single copy of the mutation. One of the wells (well 5 ) showed a S.N ratio of greater than 120, and may have received 2 copies of the mutation. These results are consistent with those observed for the KRAS K12R mutation.

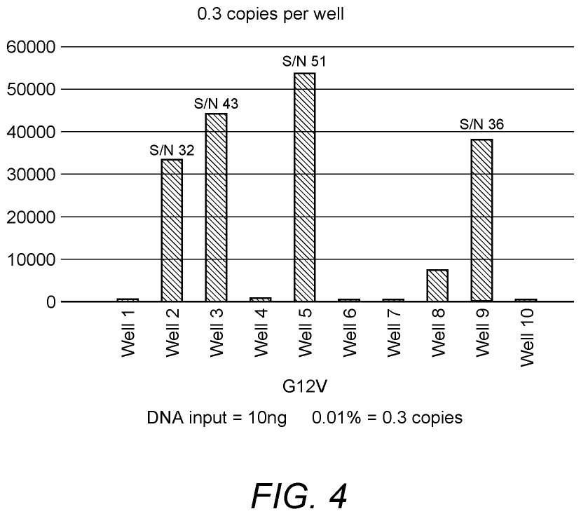

FIG. 4 shows results from ssPCR studies similar to those shown in FIGS. 2 and 3 (i.e., a mean of 0.3 copies of mutant DNA per test well), but were performed with samples containing 0.01 % KRAS G12V mutation relative to corresponding wild type DNA. Under these conditions 6 of 10 test wells show only background or near background luminescence, indicating that no mutant DNA is present. Four test wells show a clearly distinctive and detectable S/N ratio of greater than 30 from samples containing only a single copy of the mutation. These results are consistent with those observed for the KRAS K12R and KRAS G12A mutations, showing that these surprisingly sensitive results are not mutation dependent.

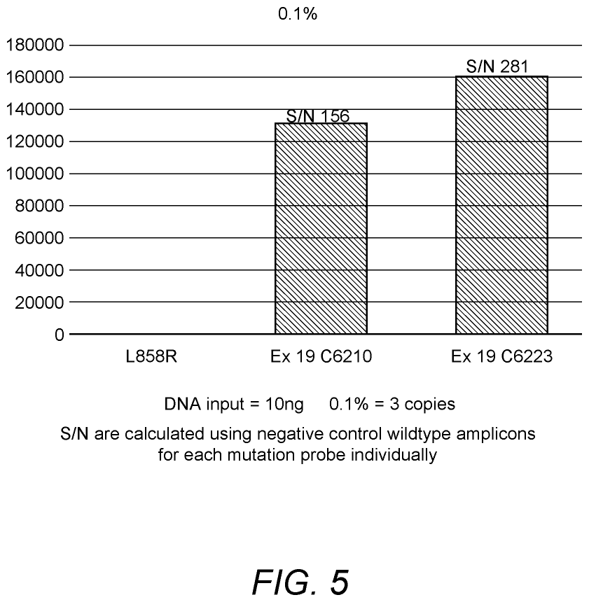

Studies were also performed using mutations at the EGFR locus. FIG. 5 shows the results of ssPCR studies performed using samples containing the EGFR exon 19 deletion at 0.1 % relative to corresponding wild type DNA. Samples contained 10 ng of DNA, for an average of 3 copies of mutant DNA per test well. Results shown are an average of the results from 3 test wells. Surprisingly, at this low frequency the S/N ratio is greater than 150.

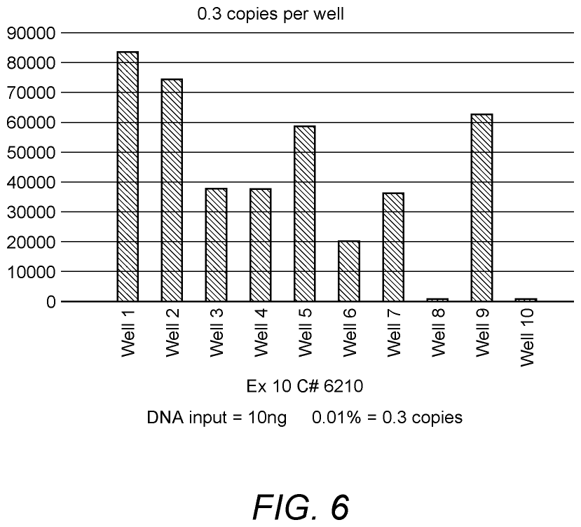

FIG. 6 shows results from ssPCR studies of samples containing the EGFR Exon 19 Del C#6210 mutation at 0.01 % relative to corresponding wild type DNA in the sample. Each sample containing 10 ng of DNA, providing a mutant copy number of 0.3 per well. Accordingly, it is to be expected that of the 10 wells tested some will not include any mutant DNA. Under these conditions 2 of 10 test wells show only background or near background luminescence, indicating that no mutant DNA is present. Many test wells show a clearly distinctive and detectable signal relative to negative wells. These results are consistent with those observed for the KRAS K12R, KRAS G12A, and KRAS G12V mutations, demonstrating that these surprisingly sensitive results are neither mutation nor locus dependent.

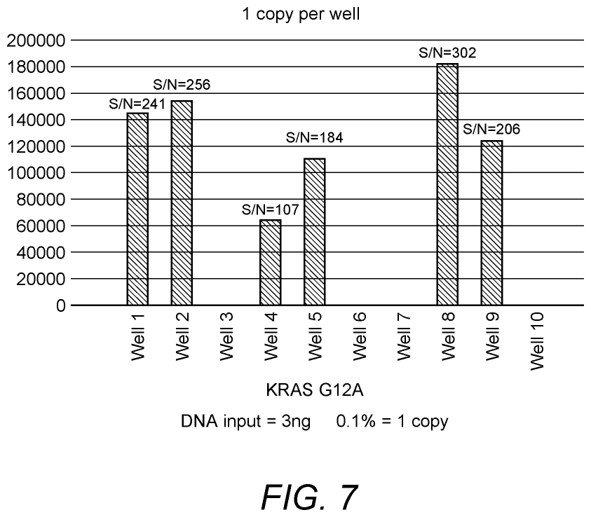

Additional ssPCR studies were performed using smaller amounts of DNA. FIG. 7 shows the results of studies performed using samples containing only 3 ng of DNA containing 0.1 % EGFR C6223 mutation relative to corresponding wild type DNA. Results from 10 individual test wells are shown. It should be appreciated that this corresponds to an average of 1 copy per test well, so it is to be expected that some test wells will not include mutant DNA. Several wells did not include mutant DNA and show background levels of luminescence. Surprisingly, robust S/N ratios of greater than 100 were observed in remaining wells despite the small amount of DNA tested and the low frequency of the mutation.

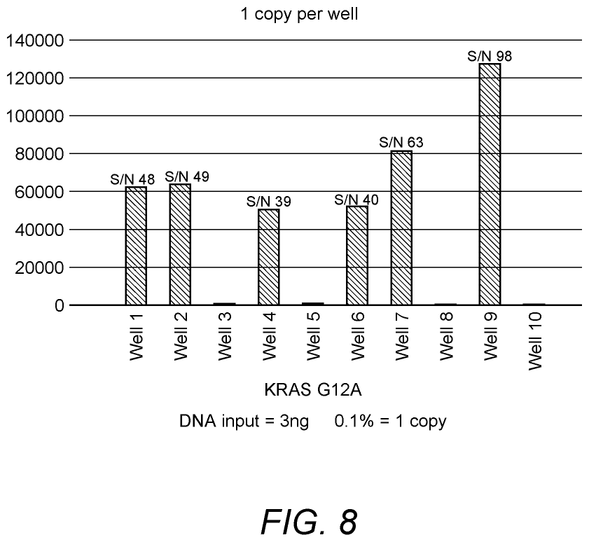

FIG. 8 shows the results of ssPCR tests performed using the KRAS G12A mutation under conditions corresponding to those used in FIG. 7 (3 ng of DNA per well, 0.1 % mutant DNA). Results from 10 individual test wells are shown. Several wells did not include mutant DNA and show background levels of luminescence. Surprisingly, robust S/N ratios of equal to or greater than about 40 were observed in remaining wells despite the small amount of DNA tested and the low frequency of the mutation. Accordingly, results from the inventive method at low DNA content and low mutation frequency are not locus dependent.

Overall, it is apparent that the ssPCR method provides reliable detection of mutations at low frequencies (0.1 % or less relative to corresponding wild type DNA present in the sample), and can do so reliably under a variety of DNA conditions where as few as 1-5 copies of mutated DNA are present in a vast excess of wildtype DNA (e.g., 3, 10, 50, 100, 333 ng hgDNA). It should be appreciated that this corresponds to conditions where PCR amplification typically requires a large number (e.g. 25 to 30 or more) to demonstrate a typical logarithmic growth curve.

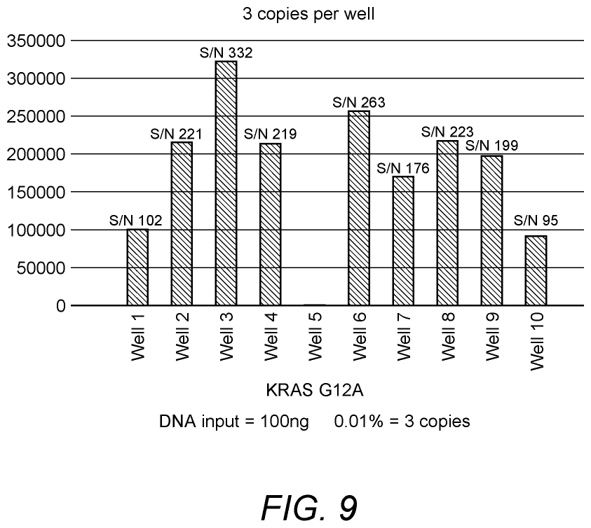

Under some conditions the amount of DNA available for testing is not limiting. Accordingly, Inventors characterized performance of the ssPCR method using large amounts of DNA per test sample. FIG. 9 shows results from testing 10 samples containing 100 ng per test sample, where the samples contain 0.01 % KRAS G12A mutation relative to corresponding wild type DNA in the sample. Under these conditions a typical sample would be expected to include 3 copies of the mutant DNA. Despite a mutation frequency of only 0.01 %, surprisingly 9 of 10 test wells showed a robust and readily detectable S/N ratio of equal to or greater than about 100.

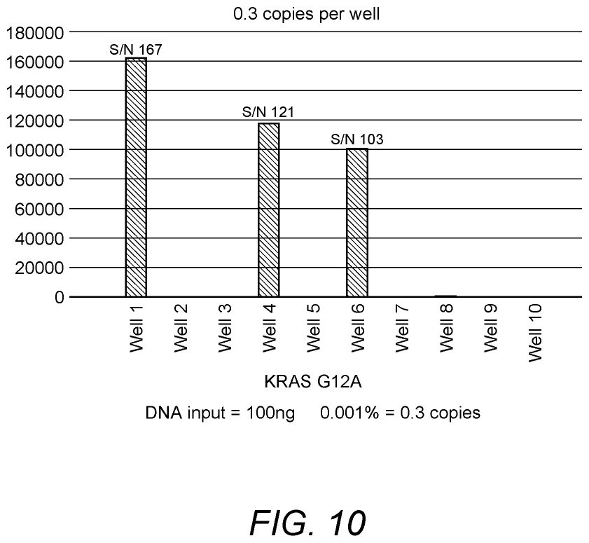

FIG. 10 shows the results of ssPCR studies similar to those shown in FIG. 9 (i.e. 100 ng DNA per test sample), but with the KRAS G12A mutation present at 0.001 % relative to corresponding wild type DNA. This corresponds to an average of 0.3 copies of the mutation per sample, against a background (99.999 %) of 100 ng of wild type DNA. Accordingly, it is to be expected that some test samples will not include the mutation. As shown, a number of test samples show background levels of luminescence consistent with the absence of mutant DNA. Surprisingly, single copies of the mutation show robust and readily detectable S/N ratios of greater than 100 using the inventive method.

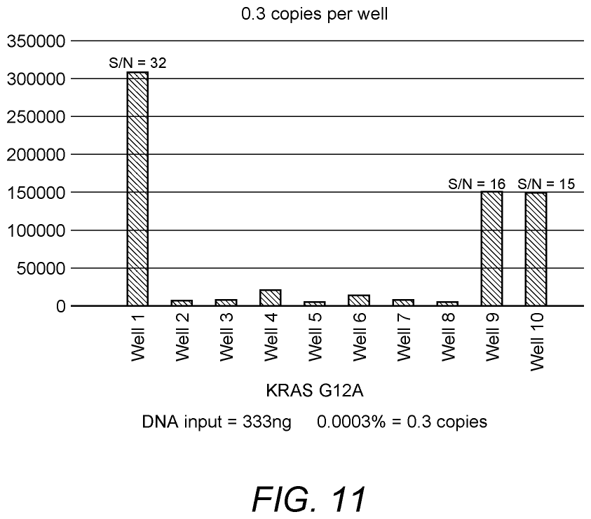

Inventors also characterized performance of the ssPCR method in test samples containing larger amounts of DNA. FIG. 11 shows the results of studies performed on 10 test samples each containing 333 ng of DNA, which included 0.0003 % KRAS G12A mutation relative to corresponding wild type DNA. This corresponds to an average of 0.3 copies of the mutation per sample. Accordingly, it is to be expected that some samples will not include mutant DNA. 7 out of 10 wells show background levels of luminescence, and presumably do not include mutant DNA. Surprisingly, 3 wells show S/N ratios of greater than 15. Inventors contemplate that a S/N ratio of 3.5, 5, 10 or greater is readily distinguishable from background and is readily detectable. Accordingly, the inventive method can provide reliable detection at mutation frequencies as low as 0.0003 %.

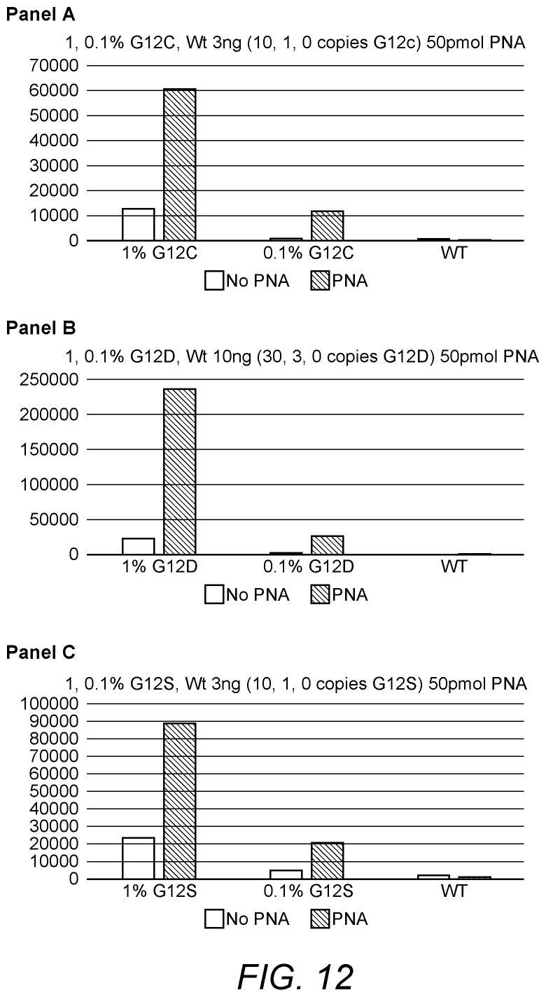

Additional KRAS mutations were also characterized by ssPCR. FIG. 12 shows results from studies performed for detection of additional KRAS mutations. Panel A of FIG. 12 shows typical results from 3 ng total DNA samples that include 10, 1, or no copies of KRAS G12C mutation. Panel B of FIG. 12 shows typical results from 10 ng total DNA samples containing 30, 3, or no copies of KRAS G12D mutation. Panel C of FIG. 12 shows typical results from 3 ng total DNA containing 10, 1, or no copies of KRAS G12 S mutation. Remaining DNA in all instances was KRAS wild type, amplification of which was inhibited by use of a PNA clamp. In all instances very low copy (e.g. 1 to 3 ) copies of the mutation were readily detectable in the presence of a large excess of the wild type sequence.

Tables 5, 6, and 7 show results of studies using mutant Allele-Specific PCR primer (AS-PCR) designs that contain a single 3′ mismatch on the wildtype template to achieve suppression of the wildtype. Samples contained 10 ng of DNA, which included down to 0.1 % KRAS G12X mutation (M). AS-PCR specific for KRAS mutants G12A, G12C, G12D, and G12R were amplified on wildtype human genomic DNA samples (Promega p/n G3041 ) and genomic DNA from engineered cell lines containing the relevant mutation at a 50 % of the genomic DNA were diluted 10-fold with Promega wildtype DNA. Each reaction contained 10 ng of genomic DNA, or approximately 3,000 copies of KRAS target. In the case of the samples containing 5 % mutant (i.e., “5 % M” in Table 5 ), there were 150 copies of each mutant in a background of 2,850 copies of wildtype KRAS target. Samples containing no mutations (i.e., “0 % M” in Table 5 ) contained approximately 3,000 copies of wildtype KRAS sequence. PCR Cycle Threshold differences between the 0 % M and 5 % M samples for each AS-PCR reaction are shown in the column “Ct Change between 5 % M and 0 % M.” In all cases, the “0 % M” Ct values were larger (data not shown) than the 5 % M samples by the difference given in this column. The ss-PCR Probe Test results are shown for samples amplified by AS-PCR, then tested using probe sequences specific for those mutations. Results are shown for the samples with 5 % Mutant (150 initial mutant copies), 1 % Mutant (30 initial mutant copies), and 0 % Mutant (wildtype DNA, 0 mutant copies).As shown in Table 5, G12A and G12R provided significant WT suppression. Table 6 shows results for mutant detection sensitivity of G12A and G12R AS-PCR primers at 0.1 % mutant levels (equivalent to 3 mutant DNA copies per sample). Some control samples that did not include the mutation displayed WT breakthrough in these samples. Table 7 shows the same data in Table 6, but with the control sample WT breakthrough sample date excluded. In this instance the method of the inventive concept is directed towards SNP detection, and is referred to as SNP-switch PCR or ss-PCR.

TABLE 5

| | Real Time PCR | | | | | | |
|---|---|---|---|---|---|---|---|
| | AS-PCR Results | | | | | | |
| | 5% | Ct Change | ss-PCR Probe | | | S/N | |
| KRAS | Ct | between | Test Results | | | in ss-PCR | |
| Mu- | Aver- | 5% M and | 5% | 1% | 0% | 5% vs | 1% vs |
| tant | age | 0% M | Results | Results | Results | 0% | 0% |
| G12A | 31.2 | 5.0 | 247926 | 245532 | 596 | 416 | 412 |
| G12A | 30.8 | 5.1 | 173335 | 172703 | 1377 | 126 | 125 |
| G12C | 33.1 | 0.5 | 45443 | 469967 | 32220 | 1 | 1 |
| G12D | 33.2 | 0.3 | 85326 | 80271 | 55723 | 2 | 1 |
| G12R | 30.0 | 10+ | 109971 | 82089 | 1164 | 94 | 71 |

TABLE 6

| | Real Time PCR | | | | |
|---|---|---|---|---|---|
| | AS-PCR | | | | |
| | Results | | ss-PCR Probe Test Results, | | |
| | | Ct Change | Including WT | | |
| | | between | Breakthrough Reactions | | |
| KRAS | 0.1% Ct | 0.1% M | 0.1% | 0% | 0.1% vs |
| Mutant | Average | and 0% M | Results | Results | 0% |
| G12A | | | 120342 | 16674 | 7 |
| G12A | | | 126683 | 16961 | 7 |
| G12A | | | 124593 | 54151 | 2 |
| G12R | | | 129345 | 23073 | 6 |

TABLE 7

| | Real Time PCR | | | | |
|---|---|---|---|---|---|
| | AS-PCR | | | | |
| | Results | | ss-PCR Probe Test Results, | | |
| | | Ct Change | excluding WT | | |
| | | between | Breakthrough Reactions | | |
| KRAS | 0.1% Ct | 0.1% M | 0.1% | 0% | 0.1% vs |
| Mutant | Average | and 0% M | Results | Results | 0% |
| G12A | | | 120342 | 3215 | 37 |
| G12A | | | 126683 | 1912 | 66 |
| G12A | | | 124593 | 4079 | 31 |
| G12R | | | 129345 | 920 | 141 |

Primers used in these studies are provided in Table 8.

TABLE 8

| | | SEQ ID |
|---|---|---|
| AS-PCR Primer | Sequence | NO. |
| KRAS G12A ARMS Fwd | CTTGTGGTAGTTGGAGCTG | 23 |
| KRAS G12C ARMS Fwd | CTTGTGGTAGTTGGAGCT | 24 |
| KRAS G12D ARMS Fwd | CTTGTGGTAGTTGGAGCT | 25 |
| KRAS G12R ARMS Fwd | CTTGTGGTAGTTGGAGCT | 26 |
| KRAS G12 Rev | TGATTCTGAATTAGCTGTA | 27 |
| | TCGTCAA | |

Accordingly, DNA primers incorporating single base pair mismatches can be used in methods of the inventive concept to suppress wild type amplification.

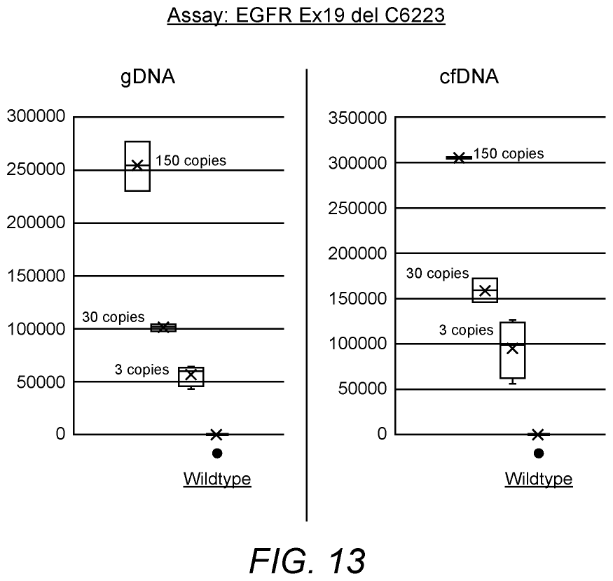

DNA samples can include intact genomic DNA and/or fragmented DNA. FIG. 13 shows the results of studies comparing the performance of detection of EGFR Ex19 del mutation in the presence of a large excess of wild type EGFR in intact genomic DNA and in fragmented DNA. As shown, as few as 3 copies of EGFR Ex19 del mutation were distinguishable over a large excess of WT EGFR in 10 ng DNA samples, regardless of whether the DNA was intact genomic DNA or fragmented.

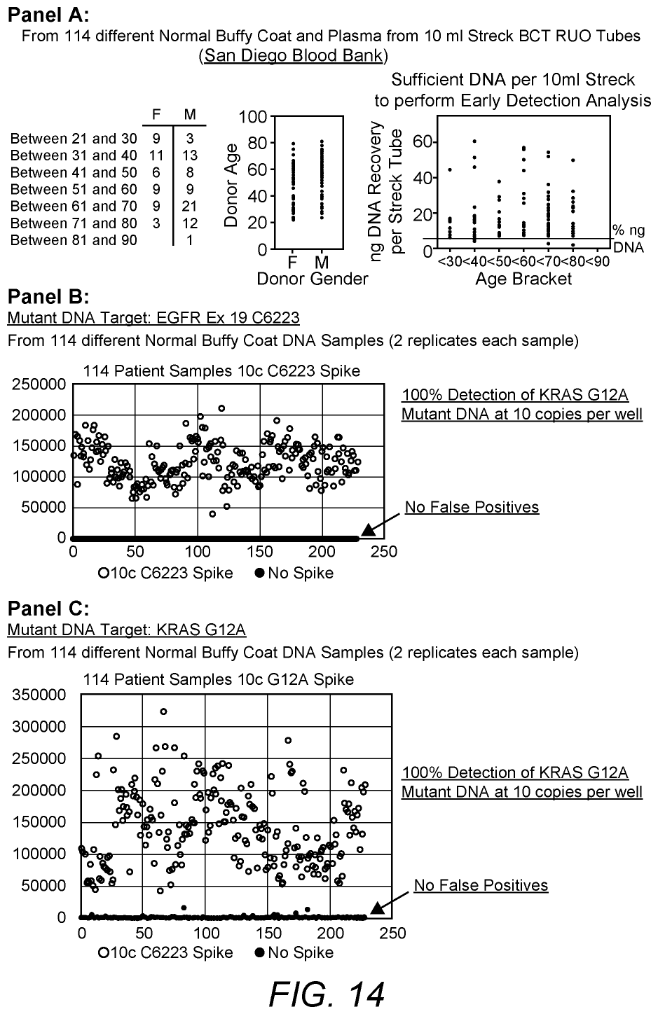

Inventors believe that methods of the inventive concept are particularly applicable to liquid biopsy diagnostics, where DNA is present in a complex biological matrix. FIG. 14 shows typical results from application of a method of the inventive concept to DNA in blood samples. Panel A of FIG. 14 shows demographic data for 114 random blood bank donors, and cell-free DNA (cfDNA) recovery from plasma extracted from a single 10 ml Streck BCT tube per donor. Panel B shows typical results from 10 ng samples obtained from the 114 donor 10 ng DNA samples using an EGRF Ex19 Del C6223 assay of the inventive concept. No positive results for this KRAS mutation were noted in the buffy coat DNA tested. Panel B of FIG. 14 also shows typical results for replicate donor DNA samples to which 10 copies of EGFR Ex19 del C6223 mutant DNA were added (i.e., spiked samples), all of which show an intensity value greater than 40,000. All of the spiked replicate samples were positive, with no false positive. Panel C of FIG. 14 shows typical results from a study similar to that shown in Panel B, but diffing in adding 10 copies of KRAS G12A mutation and directing the method of the inventive concept to same. is specific for KRAS G12A. There were no positives in the donor samples. All values above 40,000 represent the donor samples spiked with 10 copies of KRAS G12A mutant DNA. Accordingly, Inventors believe that methods of the inventive concept are highly applicable to liquid biopsy samples (e.g., buffy coat samples, cell-free DNA obtained from blood samples, etc.).

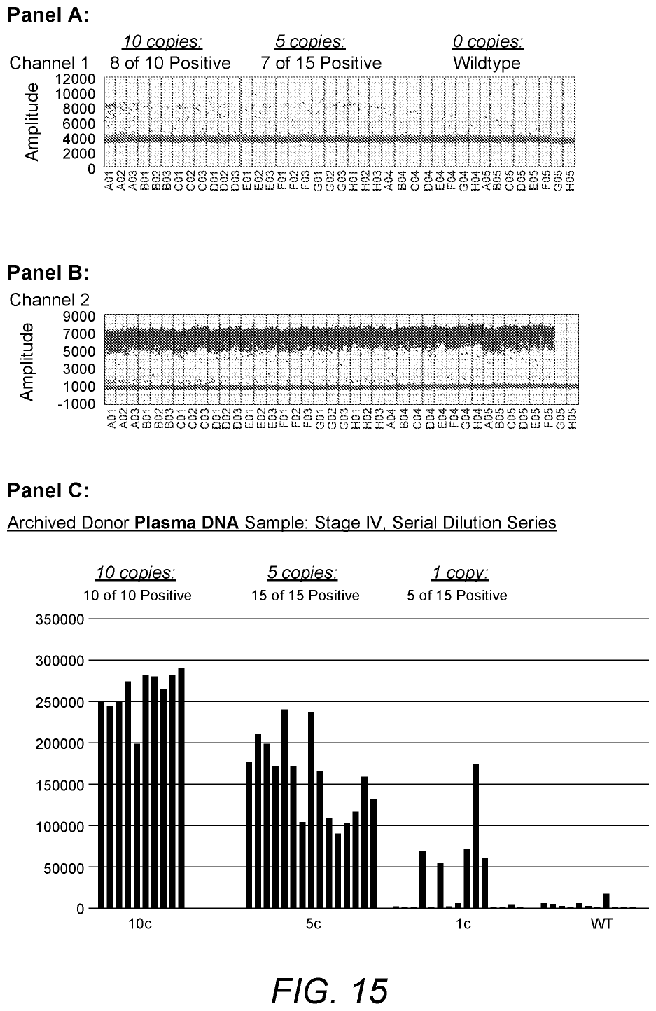

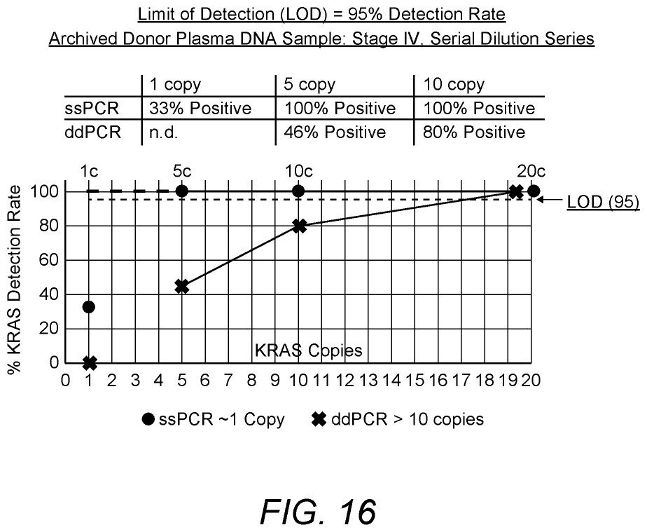

Digital Droplet PCR (ddPCR) has been used to identify rare mutations within a background of large amounts of the wild type gene. Inventors utilized a sample from a patient with Stage IV colorectal cancer to compare results of a commercial ddPCR assay (obtained from Bio-Rad) directed to KRAS G12 mutations to an assay performed using a method of the inventive concept. Results are shown in FIG. 15. A high copy level, undiluted clinical plasma sample from a patient with Stage IV colorectal cancer was initially characterized using the ddPCR assay to provide a mutant DNA copy number density. FIG. 15 shows ddPCR results and results from a KRAS mutation detection method of the inventive concept for this donor plasma DNA diluted with wildtype DNA to create samples with 10, 5, and 1 copy (ies) of mutant KRAS DNA per 10 ng of human genomic DNA. Panel A shows results from the KRAS mutation channel of ddPCR tests performed on 10 replicates of the 10 copy samples, 15 replicates of the 5 copy samples, and 10 replicates of wildtype samples (0 copies), and two samples without DNA added. As shown, 8 of 10 replicates were positive when 10 copies of the KRAS mutation were present in the 10 ng sample, and 7 of 15 replicates were positive when 5 copies of the KRAS mutation were present in the 10 ng samples. In the ddPCR method KRAS mutation positives were defined as those known mutant samples having positive results that exceeded the number of positive observed from the wildtype-only samples. Results from samples containing only a single copy of the mutant DNA were not distinguishable from wild type only samples by ddPCR (data not shown). Panel B of FIG. 15 shows results from the same samples in the wild type KRAS channel of the ddPCR method. Panel C of FIG. 15 shows typical results from a method of the inventive concept directed to the same KRAS mutation and in the same samples as those evaluated using ddPCR. Data from samples containing 1 copy of mutant DNA samples are included. The method of the inventive concept correctly identified all 10 samples containing 10 copies of the KRAS mutation and all 15 samples containing 5 copies of the KRAS mutation. The method of the inventive concept also identified 5 of 15 10 ng samples containing a single copy of the KRAS mutation, which the Inventors believe to representative of a typical distribution at this low level. This represents a considerable improvement over the commercial ddPCR method. A graphical summary of results of the commercial ddPCR KRAS kit and a KRAS detection method of the inventive concept are provided in FIG. 16.

It should be appreciated that, as shown in the above description, the inventive method is simple and straightforward to perform and that the materials and equipment required to do so are readily available. Despite its relative simplicity the inventive method is, surprisingly, capable of robust detection (e.g. S/N>15 ) at mutation rates as low as 0.0003 % relative to corresponding wild type DNA present in the sample. The Inventors contemplate that the surprisingly high S/N ratios observed at a mutation frequency of 0.0003 % indicate that reliable detection can be provided at S/N ratios of about 5 or greater at mutation frequencies as low as 0.0001 %. 0.00003 %, 0.00001 % or lower. Inventors also contemplate that additional improvements to the performance of the inventive method can be realized through the use of high fidelity polymerases in the amplification reaction.

In view of the consistent results obtained across a wide range of mutations, the Inventors believe that methods of the inventive concept are generally applicable and not limited to specific types of mutation or to specific sites.

It should be appreciated that the unexpected synergy realized in the inventive method can provide results that represent a several orders of magnitude improvement over prior art methods for detection of low frequency mutations, and can make liquid biopsies (e.g. for detection of cancer-related mutations) a viable alternative to more invasive and potentially harmful tissue biopsies. Inventors additionally contemplate that methods of the inventive concept can be utilized to detect viral, bacterial, and/or fungal pathogens present in a sample at low copy number, and to provide insights into carly development of variants (e.g. quasispeciation) in such pathogens during the course of disease.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of identifying a mutation of a wild type polynucleotide, comprising:
obtaining a sample comprising a first polynucleotide comprising the wild type polynucleotide and a second polynucleotide comprising the mutation, and wherein the first polynucleotide is present in at least a 100-fold excess over the second polynucleotide;
performing an amplification reaction on the sample while at least partially suppressing amplification of the first polynucleotide and not suppressing amplification of the second polynucleotide, using a primer pair complementary to both the first and second polynucleotides to generate an amplified first polynucleotide and an amplified second polynucleotide;
contacting the amplified sample with a probe sequence comprising a polynucleotide complementary to at least a portion of the amplified first polynucleotide and the amplified second polynucleotide, wherein the probe sequence comprises a reporter that is responsive to the mutation when the probe sequence is hybridized to the amplified second polynucleotide; and
identifying an emission from the probe sequence with a signal to noise ratio exceeding 10 when at least one copy of the second polynucleotide is present in the sample.

2. The method of claim 1, wherein the linker is coupled to a base of the probe sequence that is complementary to the second polynucleotide, and comprising a step of adding an oxidizing agent prior to identifying the emission.

3. The method of claim 1, wherein mass of the first polynucleotide and the second polynucleotide in combination is up to 300 ng.

4. The method of claim lene of claim 1, wherein the mutation is a single nucleotide polymorphism (SNP).

5. The method of claim lene of claim 1, wherein the mutation comprises a deletion.

6. The method of claim 1, wherein the mutation comprises a transposition.

7. The method of claim 1, wherein the mutation comprises a translocation.

8. The method of claim 1, wherein the mutation comprises an insertion.

9. The method of claim 1, wherein the mutation is in a KRAS gene.

10. The method of claim 9, wherein the mutation is selected from the group consisting of KRAS G12A, KRAS G12R, and KRAS G12V.

11. The method of claim 1, wherein the mutation is in an EGRF gene.

12. The method of claim 11, wherein the mutation comprises an EGRF L858R mutation, an Exon 19 deletion, a C6223 mutation, and a C#6210 deletion.

13. The method of claim 1, wherein the first polynucleotide is present in at least a 10,000 fold excess over the second polynucleotide.

14. The method of claim 1, wherein the first polynucleotide is present in at least a 100,000 fold excess over the second polynucleotide.

15. The method of claim 1, wherein the first polynucleotide is present in at least a 300,000 fold excess over the second polynucleotide.

16. The method of claim 1, wherein amplification is performed using a high fidelity polymerase.

17. The method of claim 1, wherein suppression of amplification of the first nucleotide sequence comprises application of a clamping primer comprising PNA, LNA, or XNA to the sample.
